(19) United States
(12) Patent Application Publication
(10) Pub. No.: US 2016/0086021 A1
(43) Pub. Date: Mar. 24, 2016

(54) Substance Testing Systems and Methods with Test Subject Identification Using Electronic Facial Recognition Techniques

(71) Applicant: 1A Smart Start, Inc., Grapevine, TX (US)

(72) Inventors: Wojciech Grohman, Little Elm, TX (US); Thomas Scott Caskey, Trophy Club, TX (US); Mark Bellehumeur, Frisco, TX (US)

(21) Appl. No.: 14/862,645

(22) Filed: Sep. 23, 2015

Related U.S. Application Data
(60) Provisional application No. 62/054,436, filed on Sep. 24, 2014.

(51) Int. Cl. G06K 9/00; B60K 28/06; G01N 33/497; G06F 17/30

(52) U.S. Cl. CPC G06K 9/00288; G06F 17/30244; B60K 28/063; G01N 33/4972

(57) ABSTRACT

A method for identifying a test subject being tested for consumption of a selected substance includes storing in a database of a facial recognition processing system a gallery of facial images of a set of individuals. The facial recognition processing system receives a facial image of a test subject being tested for consumption of the selected substance and compares the received facial image with at least some of the facial images in the stored gallery by executing a facial recognition algorithm. In response to a match between a facial image in the stored gallery and the received facial image having a predetermined level of match certainty; the facial recognition processing system identifies the test subject. In response to a failure to obtain a match between a facial image in the stored gallery and the received facial image having a predetermined level of match certainty, the facial recognition processing system flags the received facial image for subsequent manual analysis.

### CROSS-REFERENCE TO RELATED APPLICATION

The present application claims the benefit of U.S. Provisional Patent Application Ser. No. 62/054,436, filed Sep. 24, 2014, which is incorporated herein by reference for all purposes.

### FIELD OF INVENTION

The present invention relates in general to substance use testing techniques, and in particular to substance use testing systems and methods with test subject identification using electronic facial recognition techniques.

### BACKGROUND OF INVENTION

Sobriety testing, which includes testing for both alcohol and illegal drugs, has taken a prominent role in ensuring a safe and efficient society. For example, ignition interlocks on vehicles have proven their worth in preventing intoxicated drivers from entering the roadways and causing serious, including fatal, accidents. Sobriety testing has also allowed authorities, such as courts and law enforcement agencies, to monitor compliance with court-ordered restrictions imposed on persons having committed alcohol or drug related offenses. Among other things, with the availability of reliable sobriety testing systems, such offenders can continue travel to work, school, or rehabilitation and thus contribute to society, rather than be a burden.

Attempts to circumvent these sobriety testing systems is significant problem, with regards to both vehicle sobriety interlock systems and fixed sobriety test systems used at home and in the workplace. For example, an intoxicated driver might try to circumvent a breathalyzer-based sobriety testing system by introducing air from an air compressor, compressed air canister, balloon, or other source of intoxicant-free air. Alternatively, a monitored individual may attempt to have a substance-free individual take the test in their place.

While a number of anti-circumvention techniques suitable for use in sobriety testing systems are known in the art, these techniques are subject to a number of significant limitations. For example, some known anti-circumvention techniques used with breathalyzer-based systems require that the person being tested manipulate the breath air flow into the test apparatus. However, these techniques can be difficult for the test subject to master, are often inaccurate, and do not provide a positive identification of the individual actually taking the test.

### SUMMARY OF INVENTION

According to one representative embodiment of the principles of the present invention, a method is disclosed for identifying a test subject being tested for consumption of a selected substance, which includes storing in a database of a facial recognition processing system a gallery of facial images of a set of individuals. The facial recognition processing system receives a facial image of a test subject being tested for consumption of the selected substance and compares the received facial image with at least some of the facial images in the stored gallery by executing a facial recognition algorithm. In response to a match between a facial image in the stored gallery and the received facial image having a predetermined level of match certainty, the facial recognition processing system identifies the test subject. In response to a failure to obtain a match between a facial image in the stored gallery and the received facial image having a predetermined level of match certainty, the facial recognition processing system stores the associated pattern match score and flags the received facial image for subsequent manual analysis.

According to another representative embodiment of the present principles, a method of controlling a vehicle sobriety interlock system is disclosed, which includes continuously taking a facial image of a vehicle operator with a camera forming a portion of a vehicle sobriety interlock system. A processing algorithm determines, based on the pictures taken, if there is a person present who is attempting to take a test. If a determination is made that such person is in the position to take the test, a substance testing device forming a portion of the vehicle sobriety interlock system is activated by the processing system for testing the vehicle operator for consumption of a selected substance.

According to a further embodiment of the present principles, a method is disclosed for testing for the use of a selected substance by an individual, which employs facial detection to minimize the possibility of circumventing the test system through the use of an artificial source of substance-free air. In particular, during a predetermined test time window, an air sample is collected through an intake of a breath testing unit capable of measuring an amount of the selected substance in the air sample. During the collection of the air sample, an image is taken of an area proximate the intake of the breath testing unit with a camera. A first processor processes measurement data received from the breath testing unit to determine whether the amount of the selected substance in the air sample exceeds a predetermined threshold. A second processor processes image data received from the camera with an algorithm capable of detecting a human face from the image data with a predetermined degree of confidence. If the amount of the selected substance in the air sample exceeds the predetermined threshold, the test is recorded as failed. If the amount of the selected substance in the air sample does not exceed the predetermined threshold and a human face is not detected in the image data received from the camera, the test is recorded as failed. (Depending on the embodiment, the first and second processors could be discrete processors disposed on two separate integrated circuits, or could be implemented by a single processor unit built into a single integrated circuit, as required by other engineering trade-offs in the design of the test unit.)

In other words, certain embodiments of the present principles provide for computerized facial detection to determine whether a human is about to take a substance use test, rather than a device for circumventing the system. If the system is unable to detect that a human is prepared to take the test, the test does not proceed.

In other embodiments, a computerized facial recognition system is provided for identifying an individual taking a test for the use of a substance such as alcohol, illicit drugs, cannabis, and unauthorized prescription drugs. If the computerized facial recognition system is unable to identify the test subject with a certainty above a selected predetermined threshold, the system flags the image of the test subject for subsequent manual review by a human. By varying the certainty threshold, the administrator of the substance test can tradeoff between minimizing the need for human review and the need to minimize undetected misuse or circumvention of the substance test.

The principles of the present invention are applicable to both mobile testing systems, including vehicle sobriety interlock systems, and fixed systems, such at those used at home and in the workplace. In addition to helping ensure that the proper individual is taking the substance test, these principles also provide for a positive identification of a test subject who fails the substance test.

### BRIEF DESCRIPTION OF DRAWINGS

For a more complete understanding of the present invention, and the advantages thereof, reference is now made to the following descriptions taken in conjunction with the accompanying drawings; in which:

FIG. 1A is a diagram of a portion of the interior of a vehicle including a sobriety interlock system suitable for demonstrating one application of the principles of the present invention;

FIG. 1B is a high level functional block diagram of the exemplary sobriety interlock system utilized in the application shown in FIG. 1A;

FIG. 2 is a more detailed functional block diagram showing the primary subsystems of the handheld unit shown in FIG. 1B;

FIG. 3 is a high-level block diagram of a sobriety testing system using electronic facial recognition to positively identify an individual being tested for consumption of a selected substance, such as alcohol, illicit drugs, cannabis, or prescription drugs;

FIG. 4 is a flow chart of a general procedure for identifying an individual being tested for the consumption of the selected substance using electronic facial recognition;

FIGS. 5A and 5B are a flow chart of a particular procedure for identifying a vehicle operator being tested for consumption of the selected substance using electronic facial recognition;

FIG. 6 is a flow chart of a procedure for detecting the presence of a vehicle operator being tested for consumption of the selected substance using electronic facial detection;

FIG. 7 is a functional block diagram of an exemplary stand-alone sobriety testing system including a breath testing unit for detecting the use of a selected controlled substance by an individual (test subject) and an electronic facial detection system for minimizing the potential for circumventing the breath testing unit through the use of an artificial source of air free of the controlled substance;

FIGS. 8A and 8B are a flow chart of a procedure, suitable for use in the system of FIG. 7, which uses electronic facial detection to minimize the potential for circumventing breath testing; and

FIG. 9 is a diagram of a representative report generated by a sobriety testing system associated with the device of FIG. 7 during the execution of the procedure shown in FIG. 8.

### DETAILED DESCRIPTION OF THE INVENTION

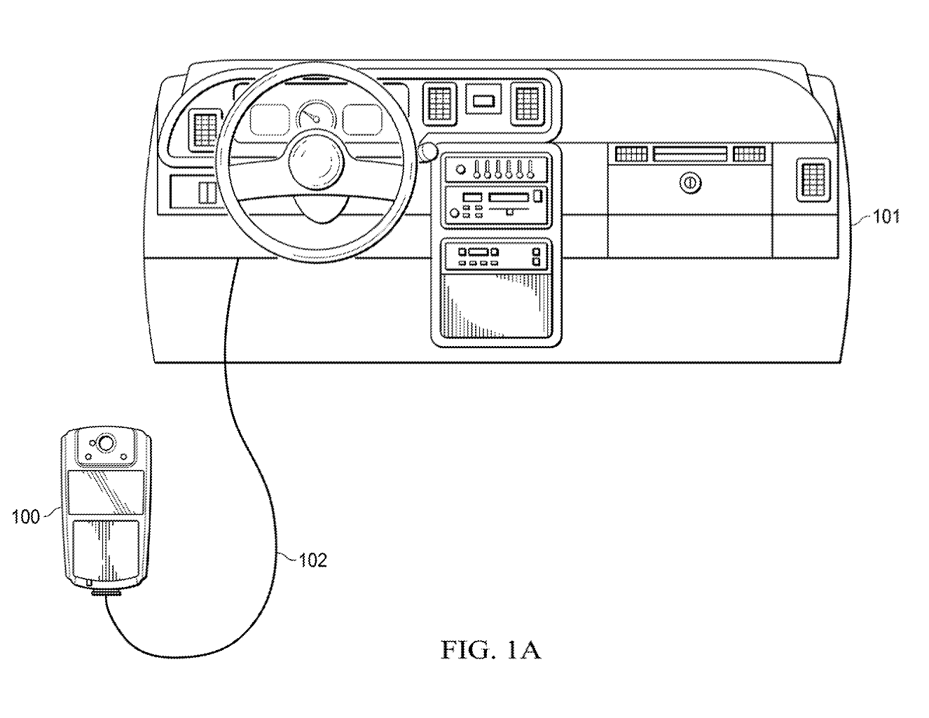

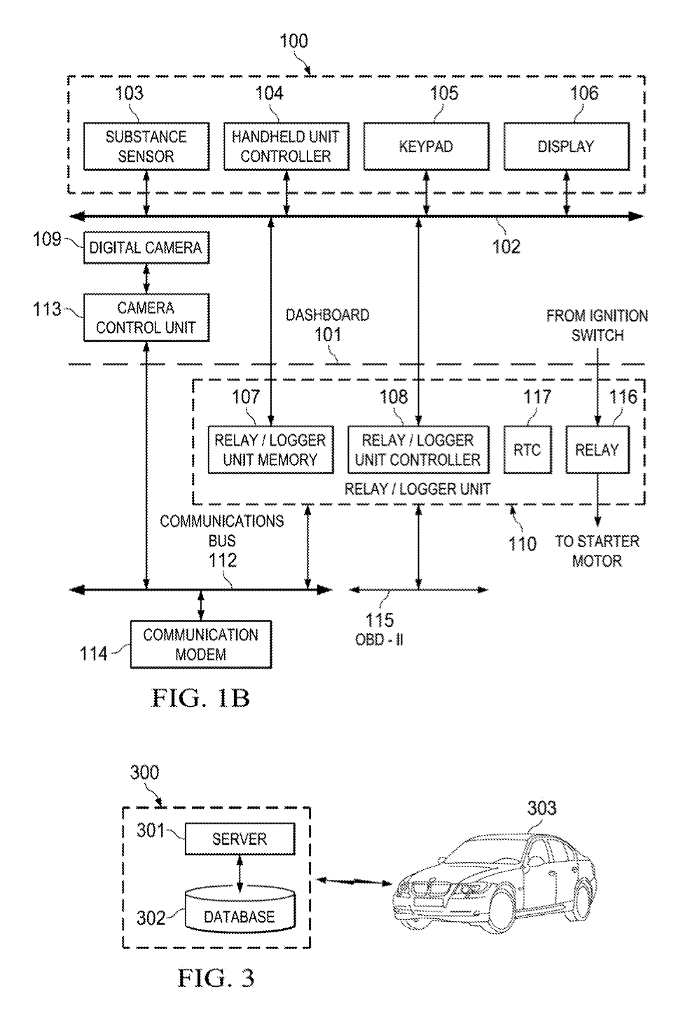

The principles of the present invention and their advantages are best understood by referring to the illustrated embodiment depicted in FIGS. 1-9 of the drawings, in which like numbers designate like parts. For discussion purposes, these principles will be described in conjunction with an alcohol breath testing system; however, the systems and methods described below are equally applicable to other types of sobriety testing systems, including those designed to test for other types of intoxicants and controlled substances (e.g., marijuana).

FIG. 1A is a diagram showing a portion of the interior of a motor vehicle in the area of the dashboard. A handheld breath alcohol testing unit 100 is connected to electronic circuitry behind vehicle dashboard 101 (see FIG. 1B) through a cable 102. Generally, a person attempting to start the vehicle must provide a breath sample to handheld unit 100, which tests for deep-lung breath alcohol content, deep-lung alcohol content being directly proportional to blood alcohol concentration and thus intoxication level. If the person being tested passes the breath alcohol test, the interlock system allows the vehicle to start. On a test failure, the interlock system disables the vehicle ignition system and the vehicle is rendered inoperable.

FIG. 1B is a high level functional block diagram of the overall interlock system. Handheld unit 100, which is discussed in detail below, includes a substance sensor 103, which in the illustrated embodiment is a fuel cell alcohol sensor, a handheld unit controller 104, a keypad 105 for data entry, and a display 106.

Handheld unit 100 electrically communicates through cable 102 with electronics behind dashboard 101. The electronics behind dashboard 101 include relay logger unit 110 with its memory 107 and relay/logger unit controller 108. Relay/logger unit memory 107, which is preferably solid state memory, such as Flash memory, stores the results of tests performed by handheld unit 100 for periodic retrieval and review by authorities monitoring the driver for compliance with any conditions or restrictions imposed on the driver. In addition, relay/logger unit memory stores the firmware controlling the operation of relay/logger unit controller 108.

Relay/logger unit controller 108, operating in conjunction with handheld unit 100, controls the operation of the vehicle in response to the outcome of a given test. As known in the art, the ignition system of a vehicle can be controlled in any one of a number of ways, including enabling or disabling relays providing power to the starter motor or sending enable or disable commands to one or more on-board computers. In the illustrated embodiment, relay/logger unit controller 108 controls a relay 116, which in turn controls the flow of electrical current between the vehicle ignition switch and the starter motor. Relay/logger unit controller 110 may also be used to generate visible or audible warnings in the event of a failed test, for example, causing the horn to sound or the headlights to flash.

A digital camera 109 or similar imaging device is also preferably provided to allow for positive identification of the person taking the breath test through handheld unit 100. The images taken by digital camera 109 are preferably stored in relay/logger unit memory 107 and/or the memory associated with the Camera Control Unit 113 for retrieval and review by the monitoring authorities. As discussed further below in detail, digital camera 109 can also take digital pictures that are then uploaded to a central server and database system for use in identifying the vehicle operator using electronic facial recognition techniques.

Advantageously, digital camera 109 reduces the possibility of a restricted or intoxicated driver of circumventing the interlock system by having a substitute person providing the breath sample to handheld unit 100. In the illustrated embodiment, digital camera 109 operates in conjunction with a camera control unit 113, which communicates with relay/logger unit controller 108 via an RS-485 standard bus 112.

Also operating off of RS-485 bus 112 is a cellular telecommunications modem 114, which allows relay/logger unit controller 108 to wirelessly send alerts to the authorities in the event of a failed test the detection of a controlled substance) or transmit logged information within relay logger unit memory 107 to the monitoring authorities, whether or not an intoxicated driver has been detected. Cellular telecommunications modem 114 particularly provides a preferred path for transmitting digital picture data from digital camera 109 to a central server and database system for vehicle operator identification.

In one particular embodiment, handheld unit 100, relay/logger unit memory 107, relay/logger unit controller 108 communicate, either in whole or in part, with the OBD-II diagnostic system 115 standard on most motor vehicles. The OBD-II system provides another efficient mechanism by which monitoring authorities can access the data stored within relay/logger unit memory 107 through a standard OBD-II port and associated test equipment. In addition, the OBD-II also allows for vehicle operating data to be recorded and stored within relay/logger unit memory 107 for correlation with the results of sobriety testing performed through handheld unit 100.

The OBD-II diagnostic system also provides a communications path for transmission of command and control signals from relay/logger unit controller 108 to various electronics and electrical systems within the vehicle. These command and control signals can be used by interlock system controller 104 to disable the vehicle in response to a failed intoxication test.

In the illustrated embodiment, relay/logger unit controller 108 includes a microprocessor or microcontroller, such as a Renesas R5F3650NDFB or similar device. A real time clock 117, such as a Seiko S-35390A, operating in conjunction with relay/logger unit controller 108, tracks the date and time.

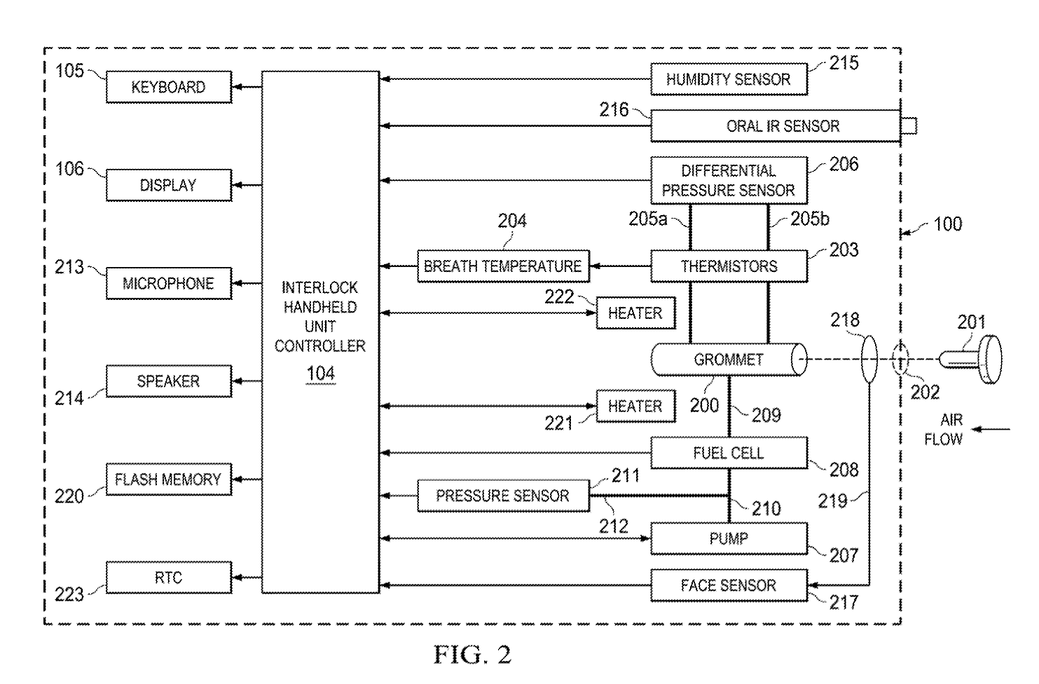

FIG. 2 is a more detailed functional block diagram of the primary subsystems within handheld unit 100 in a preferred embodiment of the principles of the present invention. In this embodiment, interlock system controller 104 is a Renesas R5F3650NDFB processor operating in conjunction with firmware stored in Flash memory 220. For clarity, interface devices, such as the analog to digital converters (ADCs) interfacing the various blocks with controller 104, and auxiliary subsystems, are not shown in FIG. 2.

A cylindrical grommet 200 receives a disposable mouthpiece 201 through an aperture 202 through the front panel of the case of handheld unit 100. Air introduced by a user (i.e., the human test subject) through mouthpiece 201 generally passes through cylindrical grommet 200 and passes out an aperture through the unit rear panel.

As air flow passes through grommet 200, a set of at least one thermistor 203 and associated breath temperature measurement circuitry 204 measure breath temperature. Breath temperature is one parameter useful for detecting attempts to circumvent an alcohol breath test.

A pair of tubes 205a-205b tap the airflow through grommet 200 to a differential pressure sensor 206, which measures breath pressure and breath air flow rate. As known in the art, in order for an alcohol breath test to be valid, the user must provide sufficient air pressure for a sufficiently long period of time to ensure that a deep-lung air is received by the alcohol sensor. If neither of these two conditions is met, interlock system controller 104 aborts the test and the breath test functional routine is reset. One device suitable for use as differential pressure sensor 206 in the embodiment of FIG. 2 is a Sensormatic 35AL-L50D-3210 differential pressure transducer.

Once interlock system controller 104 determines that deep-lung air is being received, a pump 207 is activated to draw a sample of the air flowing through grommet 200 into a fuel cell 208. In the illustrated embodiment, the air sample is drawn through tubes 209 and 210. A pressure sensor 211 monitors the air pressure being provided by pump 207 through a tube 212. One suitable fuel cell 208 is a Dart Sensors LTD 2-MS3 fuel cell operating in conjunction with a pump 207 available from PAS International, although other commercially available fuel cells and pumps may be used in alternative embodiments. A suitable device for pressure sensor 211 is a Sensormatic 33AL-L50D-3210 pressure transducer.

Fuel cell 208 implements a well-known electrochemical process to determine the breath alcohol content of the deep-lung air sample. From the air sample, interlock system controller 104 calculates the corresponding blood alcohol concentration and determines whether the user has passed or failed the test, depending on the legal limits imposed by the given jurisdiction. In response to the test result, interlock system controller 104 sends commands to vehicle electronics/electrical system 108 to enable or disable the vehicle ignition system. The results of the test are also recorded within relay/logger unit memory 107 for access by the monitoring authorities.

The user interacts with system controller 104 through keypad 105 and display 106, which allow the user to receive prompts and initiate a test in anticipation of starting the vehicle. Keypad 105 is understood to be a physical set of at least one push down key, a set of soft-keys on the device's touchscreen interface, or equivalent. In addition, interlock system controller 104 may periodically require retest of the user to ensure driver sobriety after initial start of the vehicle. In alternate embodiments, a microphone 213 and speaker 214 allow for control of handheld unit 100 by voice command.

In the illustrated embodiment of handheld unit 100, multiple sensors are provided for preventing circumvention of the breath test. In addition to breath temperature circuitry 204, handheld unit 100 also includes a humidity sensor 215, an oral infrared (IR) sensor 216, and a face proximity sensor 217. In the embodiment shown in FIG. 2, face proximity sensor 217 operates in conjunction with an electrode 218 disposed on the inner surface of the front panel of the case of handheld unit 100 and at least partially surrounding aperture 202. A clip 219 provides an electrical connection between the printed circuit board on which face proximity sensor circuit 217 resides and electrode 218.

Temperature can have a significant effect on the operation of handheld unit 100 at cold or very cold temperatures. Among other things, the speed of the electrochemical reaction within fuel cell 208 typically decreases with decreasing temperature. In addition, fuel cell 208 also is subject to a temperature coefficient, wherein the strength of the generated detection signal decreases with decreasing temperature. In addition, when grommet 200 is cold, condensation from the test subject's breath can adversely impact the test measurement.

In order to ensure proper breath content measurements are taken, grommet 200 is heated by a heater 222, which is, for example, one or more metallic sheets disposed around the grommet outer periphery. Similarly, a heater 221 maintains the temperature of fuel cell 208. Heater 221 may be, for example, a metallic sheet disposed against one or more of the outer surfaces of fuel cell 208 or a metal block on which fuel cell 208 sits. In embodiments of handheld unit 100 using a Renesas R5F3650NDFB microcomputer, heaters 221 and 222 are driven with pulse width modulated (PWM) signals that can be made available at certain controller input/output pins by firmware programming. In addition, the temperature of fuel cell heater 221 and grommet heater 222 are monitored and corresponding signals returned to handheld unit controller 104.

Electronic facial recognition has gained widespread use in both commercial and governmental applications, such as security and law enforcement. Generally, a digital picture (image) of a target (“probe”) individual is compared with a gallery of digital pictures stored in an electronic database using known facial recognition techniques, including those applying geometric (feature-based) and photometric (view-based) algorithms.

For example, the principle components analysis (PCA) technique normalizes the probe picture and the gallery images to line up the eyes and mouths. After compressing to remove unnecessary data, the remaining facial data of the probe and gallery pictures are decomposed into orthogonal (uncorrelated) components (“eigenfaces”). A feature vector is generated for each probe and gallery picture image by taking a weighted sum of the corresponding eigenfaces. To attempt to identify the person in the probe image, the distance between the probe image feature vector is compared with the feature vectors of at least some of the images in the gallery. The PCA process has the advantage of being capable of identifying facial features from only a small amount of input data, although it is necessary that the probe and gallery images be full frontal facial representations of the subject individuals.

Linear discriminant analysis (LDA) takes a statistical approach to facial recognition. Generally, samples of unknown classes are classified based on training samples from known classes. The LDA algorithms maximize between class variance and minimizes within class variance.

Elastic bunch graph matching (EBGM) relies on the fact that real faces have nonlinear characteristics, which result, for example, from differences in lighting, pose, and expression. Generally, a Gabor wavelet is used to project the image of a face onto an elastic grid. A series of nodes (“Gabor jets”) are then generated on the graph by convolving the pixels of the image with a Gabor filter. The Gabor filter takes advantage of the fact that edge detection using frequency and orientation is similar to the way the human visual system discriminates between facial features. To compare images, the Gabor filter response at each of the nodes created on the corresponding flexible grids are compared.

Commercially available facial recognition software is marketed and sold by a number of companies, including Microsoft, IBM, Conitech Systems GmbH, and Sensible Vision, Inc. Open source facial recognition software, such the Open Source Vision Library (OpenCV) is also available. In addition, standards for the management of facial recognition data are being developed, including ISO SC37 19794-5 for Standards for Biometric Data Exchange Formats, Facial Image Data.

FIG. 3 is a high-level functional block diagram of a electronic facial recognition system 300 embodying the principles of the present invention. Facial recognition system 300 includes a server 301 and a database 302, which together store a gallery of facial pictures of monitored individuals and execute known facial recognition algorithms, such as those discussed above. Preferably, system 300 is based upon commercially available or open source facial recognition software.

In an alternate embodiment, electronic facial recognition system 300 may be embedded into the vehicle interlock system of vehicle 303. For example, system 300 may reside within either handheld unit controller 104, relay/logger unit controller 108, camera control unit 113, or any combination of the three.

Typically, a law enforcement agency, court, or commercial sobriety testing/interlock provider will take a digital reference picture of the face each individual being monitored, for example, following a criminal offense involving alcohol or a controlled substance. These reference pictures are preferably used to populate the facial picture gallery within database 302, although other sources of facial pictures (images) can be used to populate the gallery. In the preferred embodiment the pictures of the client taken by camera 109 during the installation of substance monitoring system are used as reference images for facial recognition. This simplifies the facial recognition process as the hardware used to take the reference picture and pictures for analysis is the same.

In the illustrated embodiment, facial recognition system 300 receives facial image data from sobriety interlock system digital camera 109 via communications modem 114 and a wireless link. However, the principles of the present invention are not limited to either sobriety interlock systems or wireless communications embodiments. In alternate embodiments, the digital camera may be provided as part of a non-mobile substance testing and monitoring system for home or workplace use. In these non-mobile embodiments, the communications link can be wireless, hardwired (e.g., a hardwired Internet connection), or a combination of both (e.g., a WiFi hotspot and hardwired Internet connection.)

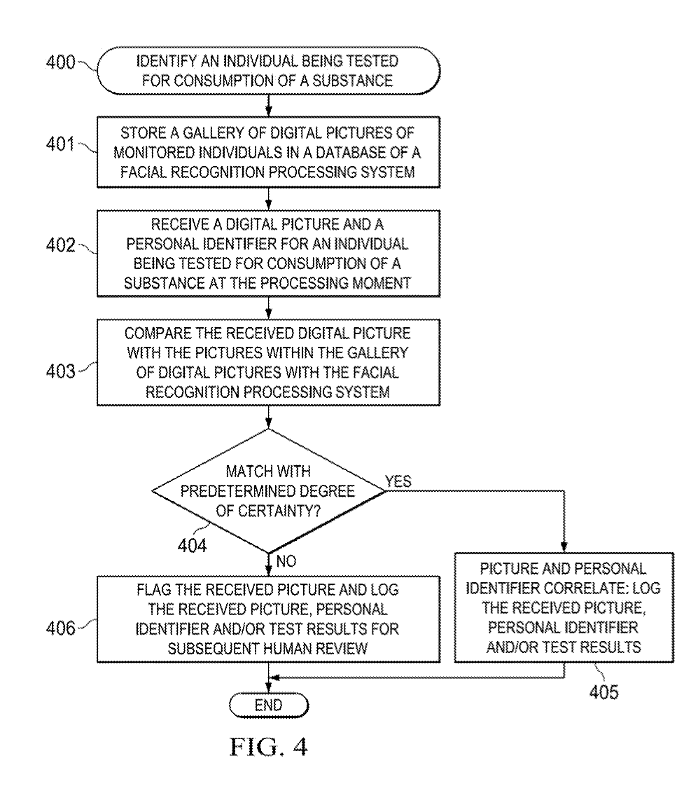

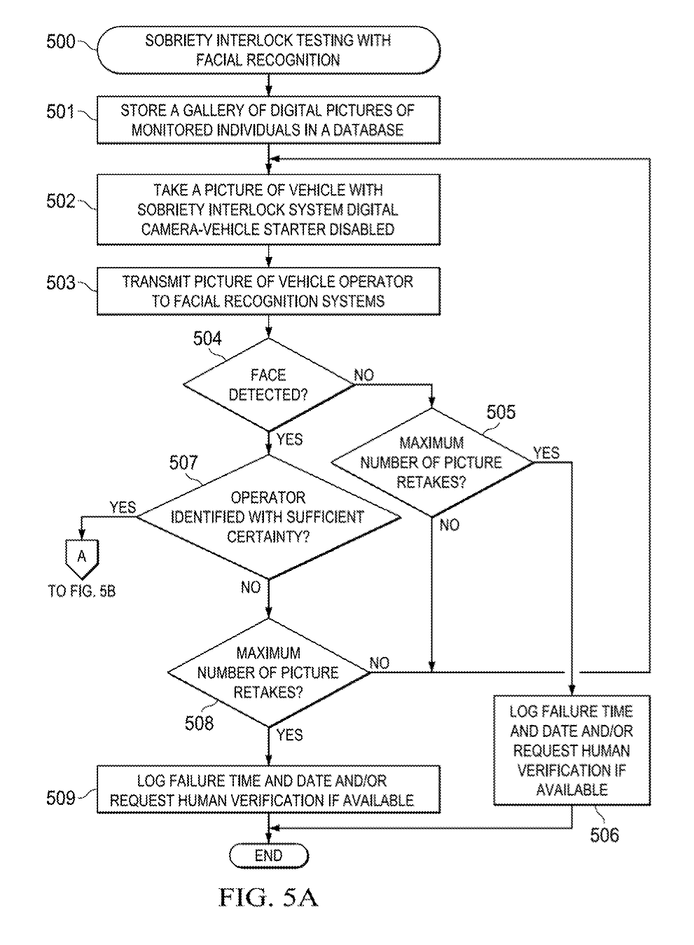

FIG. 4 is a flow chart of an exemplary Procedure 400 for identifying an individual being tested for consumption of alcohol or a controlled substance (e.g., cannabis, illicit drugs, unauthorized prescription medications). Procedure 400 can advantageously be used in both vehicle sobriety interlock systems and non-mobile substance testing systems.

At Block 401, a gallery of digital pictures (images) are stored in database 302 of system 300. As discussed above, these pictures have been collected, for example, by a law enforcement agency, court, or commercial sobriety testing/interlock provider responsible for the monitoring of individuals associated with substance-related legal offenses. A picture is taken of the test subject at Block 402 and sent to the system 300, along with a putative personal identifier (e.g., name, social security number, or other unique identifier associated with the individual undergoing the test). Server 301 then compares the received digital picture with the pictures of the gallery using facial recognition techniques (Block 403).

If at Decision Block 403, a match occurs between the picture of the test subject and a picture in the gallery, with an acceptable degree of certainty, then the personal identifier and the picture of the test subject are deemed to correlate. In this case, the individual's personal identifier, the picture of the test subject, and/or the test results are logged (Block 405). On the other hand, if no match occurs with an acceptable degree of certainty, at Block 406, the picture of the test subject is flagged and logged along with the personal identifier for the test subject and/or the test results. The flagged picture can then be manually compared by a human with the stored picture corresponding to the personal identifier to confirm that the proper individual took the substance test.

By adjusting the value of the threshold required for determining whether a match has occurred with a sufficient degree of uncertainty, the algorithm for determining whether a match has occurred with a sufficient degree of uncertainty, or both, the operator of system 300 can advantageously can trade-off the need for increased manual (human) inspection for a higher certainly level in detecting test system misuse or circumvention.

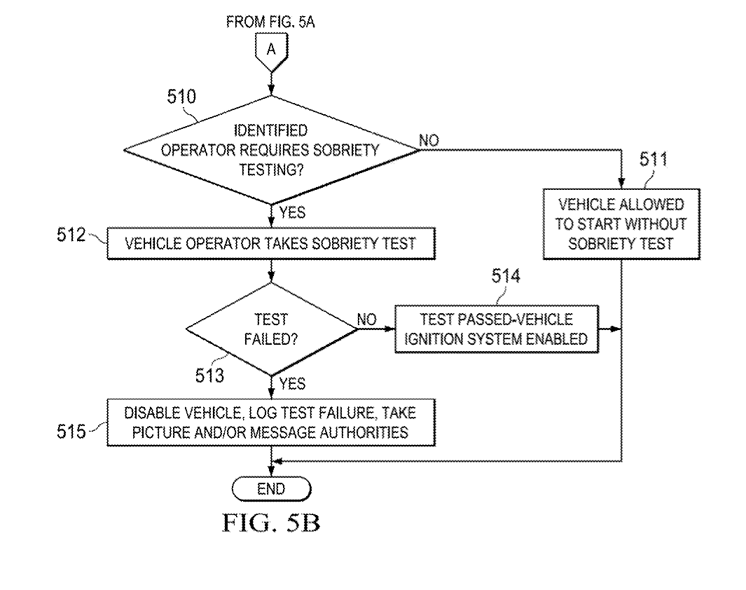

FIGS. 5A and 5B are a flow chart of a Procedure 500 for identifying the operator of a vehicle having a sobriety interlock system. Similar to Procedure 400, at Block 501 a gallery of digital pictures of monitored individuals are stored in database 302. At Block 502, a picture of the vehicle operator is taken with the digital camera of the sobriety interlock system (e.g., digital camera 109 in the system described above) prior to sobriety testing of the vehicle operator and enablement of the vehicle starter. The picture of the vehicle operator is sent to facial recognition system 300 for processing at Block 503 (e.g., via communications modem 114 in the system described above).

At Decision Block 504, a threshold determination is made as to whether the image received by the facial recognition system is generally that of a human face. (Depending on the particular processor used in the sobriety interlock system and/or the available software, this initial determination could alternatively be made within the vehicle itself). If the image does not appear to be a human face, due to an attempt to circumvent the system, improper alignment of vehicle operator's face with the camera, poor lighting, or some other factor, the vehicle operator may optionally be allowed a predetermined number of picture retakes at Decision Block 505.

If the vehicle operator avails him or herself of the maximum number of retakes, but a human face still cannot be discerned by the facial recognition system, then the failure time and date is logged and/or a request for human verification may be transmitted to system 300 at Block 506. Human verification may be made using a manual comparison of one or more of the digital pictures taken at the vehicle with the reference picture stored in system database 302, a telephone conversation, or some other means of verifying the identity of the vehicle operator with sufficient certainty.

On the other hand, if a threshold determination is made at Block 504 that a human face has been imaged by the digital camera, then at Block 507, facial recognition system 300 compares the picture taken of the vehicle operator with the pictures in the gallery stored in database 302 using full facial recognition processing. If at Decision Block 507, the vehicle operator cannot be identified with sufficient certainty, then the vehicle operator may again be allowed a predetermined number of picture retakes at Decision Block 508. If the vehicle operator is not given any picture retakes, or if identification of the vehicle operator cannot be made using the allowed number of retakes, then the failure time and date are logged and/or a request for human verification is transmitted to system 300 at Block 509.

A positive identification of the vehicle operator by facial recognition with sufficient certainty at Block 507 preferably verifies that the vehicle operator is the appropriate person taking the sobriety test. Optionally, for vehicle operators who are not monitored offenders, but who have submitted their picture into the gallery in database 302 and have agreed to any conditions imposed on their use of the vehicle (e.g., an agreement not to allow an intoxicated driver to operate the vehicle after ignition), a positive identification can allow those vehicle operator to start and operate the vehicle without requiring a sobriety test.

Specifically, if at Decision Block 510 a determination is made that the identified vehicle operator does not require sobriety testing, then at Block 511, the sobriety test is bypassed and the vehicle operator is allowed to start and operate the vehicle. On the other hand, for a monitored individual, the vehicle operator takes the sobriety test at Block 512. If the sobriety test is taken and passed, at Decision Block 513, the vehicle ignition is enabled at Block 514. Otherwise, on a failed test, the vehicle is disabled, another picture may be taken, and/or the authorities are messaged (Block 515).

In particular alternate embodiments, only recognized registered persons are required to take the sobriety test. In this case, if a person taking the test is not recognized, or the recognition score for the person taking the test is a very weak match against the reference pictures of the monitored offender, the system may still allow the person to start the vehicle at Blocks 510 and 511.

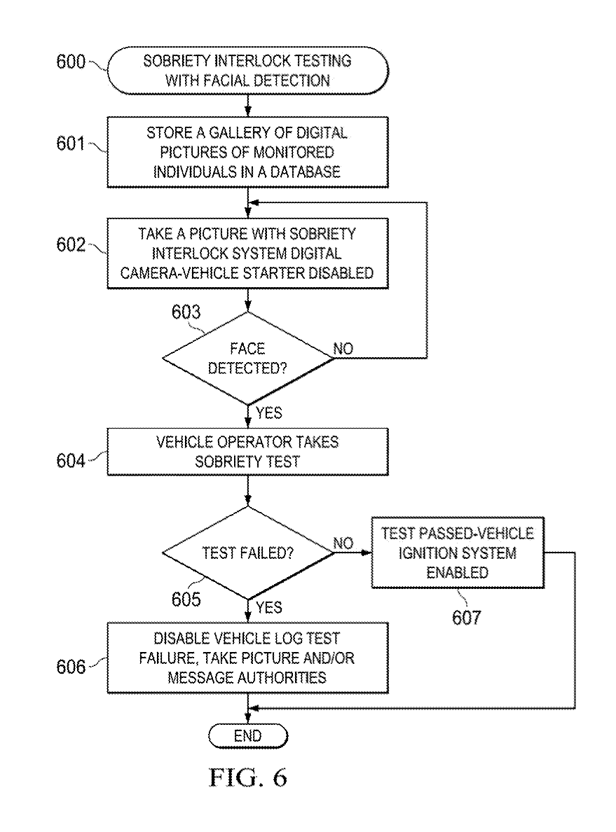

FIG. 6 is a flow chart of a Procedure 600 for detecting the presence of a vehicle operator being tested for consumption of a substance using facial detection. In contrast to facial recognition, where the identity of the vehicle operator is determined with a certain degree of certainty, facial detection only requires the detection of a human face in front of the testing system camera. Advantageously, facial recognition can be implemented using a simpler set of algorithms and a reduced amount of processing power

At Block 601, a gallery of digital pictures of monitored individuals are stored in database 302 of system 306 (FIG. 3). A picture of the then taken with the digital camera of the sobriety interlock system (e.g., digital camera 109 in the system described above) prior to sobriety testing of the vehicle operator (Block 602).

A determination is made, at Block 603, as to whether a human face was present when the picture was taken at Block 602 using a facial detection algorithm and the stored gallery of pictures. This step can be executed remote from the vehicle (e.g., using server 301 and communications modem 114 described above) or onboard the vehicle (e.g., using relay/logger unit controller 108).

If at Decision Block 603 a human face is not detected, for example because of insufficient picture quality or because an attempt has been made to circumvent the test, then Procedure 600 optionally returns to Block 602 for a re-take of the picture. In some embodiments, similar to those discussed above, the vehicle operator is allowed a certain number of re-take attempts before the system is locked and must be reset by the sobriety interlock system administrator. In other embodiments, a picture re-take may not be allowed, pending affirmative action by the sobriety interlock system administrator.

On the other hand, if a human face is successfully detected, then at Block 604 the vehicle operator takes the sobriety test. If the sobriety test is taken and passed, at Decision Block 605, the vehicle ignition is enabled at Block 607. Otherwise, on a failed test, the vehicle is disabled, another picture may be taken, and/or the authorities are messaged (Block 606).

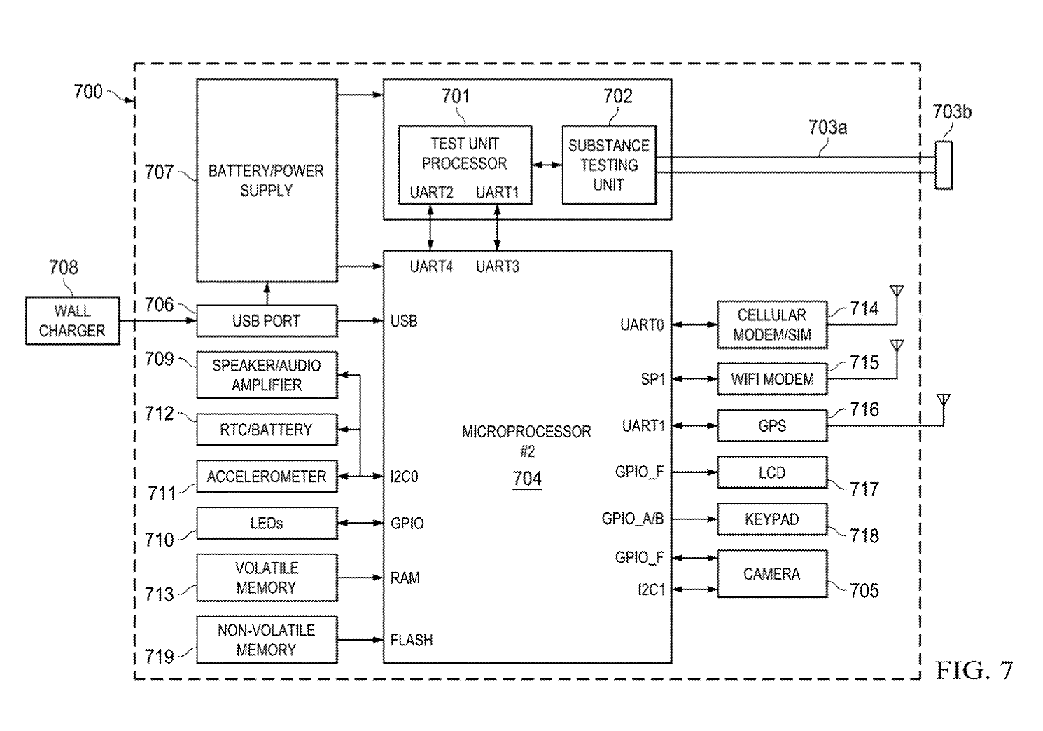

FIG. 7 is a functional block diagram of a stand-alone sobriety testing system 700 embodying the principles of the present invention. Although system 700 preferably operates in conjunction with a server and associated database, such as server 301 and database 302 of FIG. 3, for purposes of reporting test results, system 700 is otherwise a fully integrated system that performs testing on individuals for the use of a given substance, processes image data for facial detection to minimize test circumvention, and packages communicates the results.

System 700 advantageously employs facial detection to minimize the possibility of test circumvention by ensuring that a human breath sample is received, rather than substance-free air provided by artificial means, such as a balloon. System 700 is therefore particularly suitable for unsupervised at-home monitoring of an individual for the consumption of a given substance, such as alcohol, illicit drugs, cannabis, or prescription drugs, as mandated by a court or other governmental authority. However, system 700 may also be used by businesses, governmental agencies, or the like, for monitoring compliance with substance use policies.

System 700 includes a test unit processor 701, such a Renesas R5F3650NDFB microprocessor or similar device, operating in conjunction with a substance testing unit 702. In the preferred embodiment, substance testing unit 702 is similar to the breath testing system described above in conjunction with FIG. 2, and includes an appropriate fuel cell and auxiliary components, such as a pump, heaters, temperature and pressure sensors, and so on. Substance testing unit 702 receives breath samples through a breath intake conduit 703a and a mouthpiece 703b.

System 700 also includes a system processor 704, which preferably is one of the Freescale K61 family of microprocessors. For purposes of the present discussion, system processor 704 executes image data processing algorithms on the image data provided by a camera 705 to detect, with a sufficient degree of confidence, the presence of a human face in the area around mouthpiece 703b during substance testing. In the preferred embodiment, this image processing only confirms that a human is taking the test through facial detection, rather than identifying the person taking the test.

In addition, a system processor 704 controls and processes data generated from the other main functional components of system 700. For example, system processor 704 communicates with external devices through a USB port 706, which also couples electrical power from an external wall charger 708 to the system power supply and battery unit 707. System 700 also includes an audio unit 709, including a speaker and audio amplifier, indicator LEDs 710, an accelerometer 711, and a real time clock (RTC) 712, each of which operates in conjunction with system processor 704. The system memory also includes volatile 713 and nonvolatile memory 719.

System processor 704 operates in conjunction with multiple interface and communications devices including a cellular modem 714 and associated subscriber identity module (SIM), a WiFi modem 715, GPS receiver 716, LCD 717, and a keypad 718.

While the use of two separate processors 701 and 704 advantageously increases the functionality and reliability of system 700, in alternate embodiments the functionality of processors 701 and 704 may be provided by a single microprocessor 704. In such case, the sole microprocessor 704 performs functions of both units, preferably running as two separate software threads or processes within the microprocessor hardware.

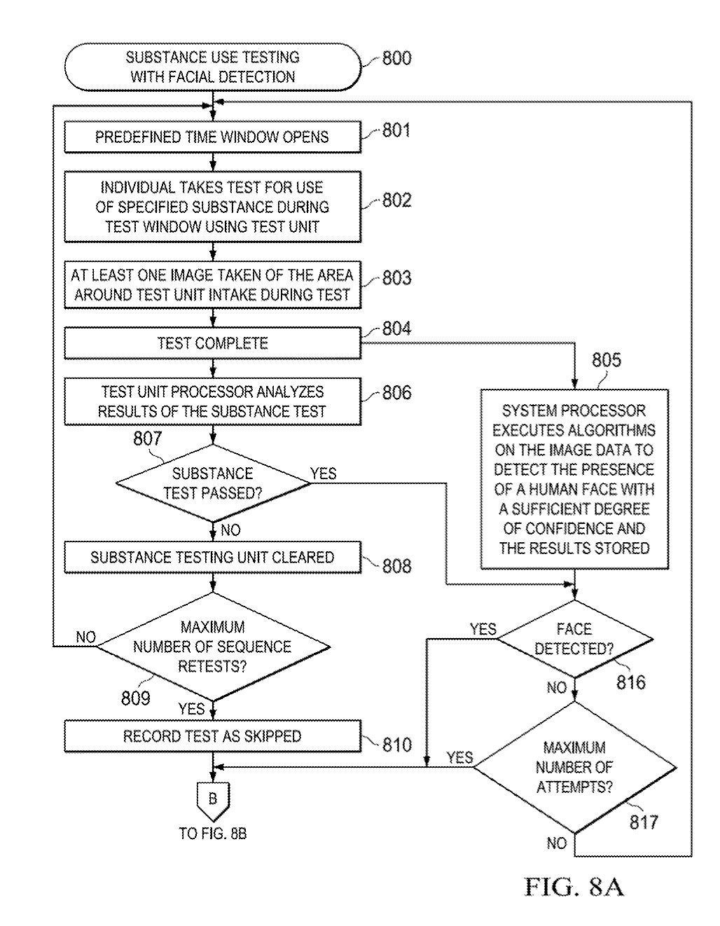

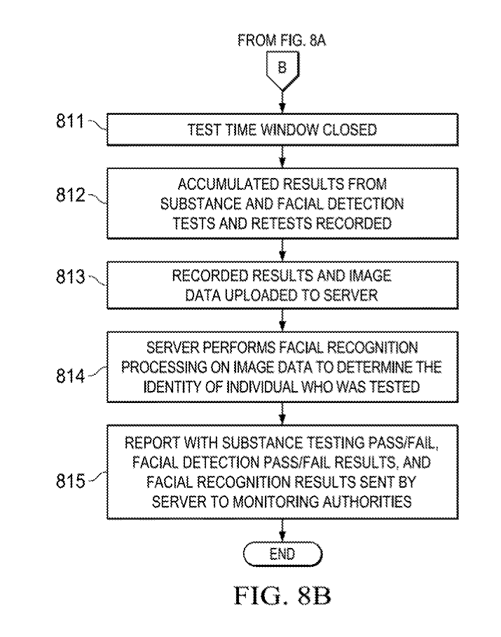

A preferred substance use testing Procedure 800 is shown in FIGS. 8A and 8B. Generally, Procedure 800 uses facial detection to minimize the possibility that a test subject will attempt to circumvent a test by using an artificial source to provide an air sample that is free of the substance whose use is being monitored. While Procedure 800 will be discussed in conjunction with system 700 described above, the hardware and/or software platforms supporting the execution of Procedure 800 may vary in different embodiments of the present principles.

At Block 801, a predefined test time window opens during which an individual subjected to mandatory substance use testing (the “test subject”) must provide a breath sample to substance testing unit 702 through mouthpiece 703b. (In the preferred embodiment, monitoring and control of the test time window is performed by system processor 704). At Block 802, the test starts and an air sample is received by system 700.

During the time air is being provided to substance test unit 702, camera 705, under the control of system processor 704, takes at least one image of objects in the vicinity of mouthpiece 703b (Block 803). If the test subject is not attempting to circumvent the test, then the image data captured by camera 705 should correlate to a human face. Typically, camera 705 will take the image approximately 1.0 to 1.5 seconds after substance testing unit 702 starts receiving positive air flow through air intake conduit 703, as measured by test unit processor 701.

At Block 804, the required air sample has been received by substance testing unit 702 and the test is complete. Typically, during a normal test without an attempt at circumvention, a sufficient air sample is obtained after 3 to 4 seconds of blowing into mouthpiece 703b by the test subject. Test unit processor 701 and system processor 704 (or two separate threads or processes within sole microprocessor 704, if only one processor is used) now respectively process in parallel the data received from substance test unit 702 and camera 705. In particular, at Block 805, system processor 704 executes algorithms on the image data from camera 705 to determine, with a sufficient degree of confidence, that a human face was proximate mouthpiece 703b during the intake of the air sample. The results of the image processing are stored. In parallel, test unit processor 701 analyzes the output from substance test unit 702 to determine the content of the breath sample (Block 806), and specifically the level of the selected substance being monitored.

If test unit processor 701 determines, at Block 807, that the breath sample contains an amount of the substance being monitored above a predetermined threshold (i.e., the test subject has failed the substance use test), then the substance testing unit 702 is cleared at Block 808 by test unit processor 701. The predetermined threshold, which could be as little as zero, is set, for example, by the monitoring authorities, the laws of the jurisdiction, or by contract between the test subject and his or her employer.

At Block 809, a determination is made by system processor 704 as to whether maximum number of allowed substance use retests has been reached. If the maximum number of allowable retests has not been expended, then Procedure 800 returns to Block 801, the test time window is re-opened, and retesting is performed by repeating the operations at Blocks 802-807.

On the other hand, if the test subject fails the current test, and the maximum number of allowed retests have been expended, then the test is recorded as “skipped” at Block 810 by system processor 704. Next, at Block 811, the test time window is closed. The accumulated results from the current substance use test and any previous retests are recorded at Block 812. These results, along with the image data from camera 705 and the results of the parallel facial detection processing operations performed by system processor 704, are uploaded to a server, such as server 301 of FIG. 3, at Block 813. In embodiments using system 700, the upload to the server is accomplished, for example, using either cellular modem 714 or WiFi modem 715.

At Block 814, the server performs facial recognition processing to identify the individual who provided the air sample. In the preferred embodiment, Block 814 is implemented using Procedure 400 discussed above in conjunction with FIG. 4, although other procedures may be used in alternate embodiments. If the facial recognition processing identifies the monitored individual as the person who provided the air sample that failed, that information can be sent to monitoring authorities for use in determining compliance with the conditions placed on that individual. On the other hand, if the facial recognition processing does not identify the individual who provided the air sample as the monitored individual, whether or not the substance test was a pass or a fail, that information can similarly be forwarded to the monitoring authorities as possible evidence of an attempt to circumvent the test (e.g., another individual has provided the air sample).

The server then takes the uploaded data and generates a report, which includes substance testing pass/fail results, facial detection pass/fail results, or both. The report is then sent electronically to the monitoring authorities directly from the server (Block 815).

Returning to Block 807, if the controlled substance is not found in the breath sample (i.e., the test subject passes the substance test), then at Block 816 a determination is made by system processor 704, using the image data processed and stored at Block 805, as to whether a human face was detected proximate mouthpiece 703b during the intake of the air sample. If a human face was detected, which confirms that a human individual took the test, then procedure advances to Block 811, the test time window is closed, and the data collection and reporting steps of Blocks 812-815 previously discussed are performed.

In contrast, if a human face is not detected at Block 815, then a determination is made at Block 816 by system processor 704 as to whether the maximum allowed number of facial detection attempts have been made (Block 817). If the maximum number of attempts has been reached, then procedure 800 again advances to Blocks 811-815. On the other hand, if the maximum number of allowed facial detection attempts have not been expended, then Procedure 800 returns to Block 801, the test time window is reopened, and retesting is performed by repeating the operations at Blocks 802-807.

Procedure 800 advantageously significantly reduces the probability that a test can be circumvented through the use of an artificial source of air free of the given monitored substance. At the same time, the use of facial detection, rather than facial recognition, reduces the complexity of the required hardware and software, which allows Procedure 800 to be adapted for stand alone substance testing systems, such as system 700. A stand alone system, such as system 700, in turn, is suitable for use in home and business environments without the need for significant in-person supervision of the test subject.

In addition, the preferred embodiment also allows for the more processing-intensive facial recognition algorithms to be executed at remote server, which advantageously minimizes the possibility of a test being circumvented by having a substitute individual (sober or intoxicated) provide the air sample.

In all of the exemplary embodiments described above, it may often be advantageous to use two separate facial detection certainty thresholds instead of one. The first, usually lower, certainty threshold is used to selectively enable system 700 or handheld unit 100 to perform a test. After the first threshold is met and system 700 or handheld unit 100 is enabled, the second, usually higher, facial detection certainty threshold is used to discern those images that require a manual review. In other words, when two certainty thresholds are used, those pictures with a certainty score below the first threshold indicate that no person was present to take a test, and those pictures with certainty scores above both the first and second thresholds indicate that there is a high probability that a person was present to take the test and therefore the pictures do not require a manual review. The manual review is then required for all pictures taken with certainty scores between the two thresholds.

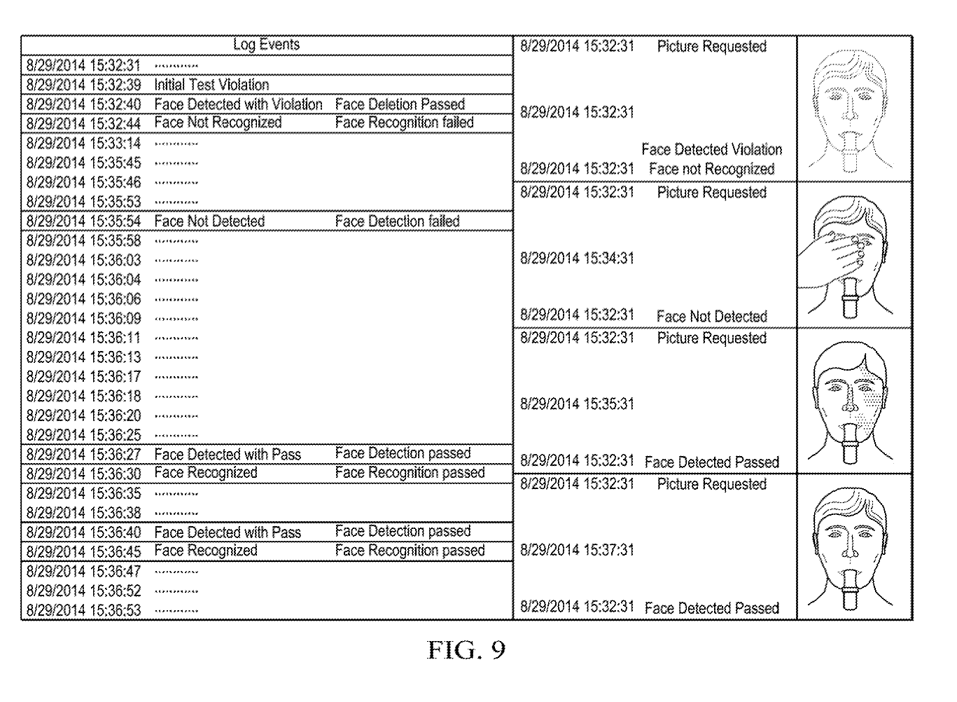

FIG. 9 illustrates an exemplary report generated by system 700 in response to periodic testing of a monitored individual. Generally, each reported instance includes the picture taken during the test along with the test date and time and the results. In the first instance, a violation (sobriety test failure) is observed, the face is detected, but the face is not recognized. In the second instance, a face is not detected. In the third and fourth instances, the face is detected, the sobriety test is passed, and the face is recognized. FIG. 9 shows only one representative report that could be generated by system 700; in actual embodiments of the present principles the form and content may change to meet the particular needs of the monitoring authorities.

Although the invention has been described with reference to specific embodiments, these descriptions are not meant to be construed in a limiting sense. Various modifications of the disclosed embodiments, as well as alternative embodiments of the invention, will become apparent to persons skilled in the art upon reference to the description of the invention. It should be appreciated by those skilled in the art that the conception and the specific embodiment disclosed might be readily utilized as a basis for modifying or designing other structures for carrying out the same purposes of the present invention. It should also be realized by those skilled in the art that such equivalent constructions do not depart from the spirit and scope of the invention as set forth in the appended claims.

It is therefore contemplated that the claims will cover any such modifications or embodiments that fall within the true scope of the invention.

What is claimed is:

1. A method for identifying a test subject being tested for consumption of a selected substance comprising:
storing in a database of a facial recognition processing system a gallery of facial images of a set of individuals;
receiving with the facial recognition processing system a facial image of a test subject being tested for consumption of a selected substance;
comparing the received facial image with at least some of the facial images in the stored gallery using a facial recognition algorithm executed by the facial recognition processing system;
in response to a match between a facial image in the stored gallery and the received facial image having a predetermined level of match certainty, identifying the test subject with the facial recognition processing system; and
in response to a failure to obtain a match between a facial image in the stored gallery and the received facial image having a predetermined level of match certainty, flagging the received facial image with the facial recognition processing system for subsequent manual analysis.

2. The method of claim 1, wherein the gallery of facial images comprises a gallery of facial images of a set of individuals being monitored by legal authorities.

3. The method of claim 1, wherein the predetermined level of match certainty is programmed into the facial recognition processing system to set a level of subsequent manual analysis and a corresponding level of test circumvention protection.

4. The method of claim 1, wherein the selected substance is selected from the group consisting of alcohol, illicit drugs, cannabis, and prescription drugs.

5. A system for identifying a test subject being tested for consumption of a selected substance comprising:
a facial recognition processing system including:
a database for storing a gallery of facial images of a set of individuals; and
a processor operable to:
receive a facial image of a test subject being tested for consumption of a selected substance;
compare the received facial image with at least some of the facial images in the stored gallery by executing a facial recognition algorithm;
in response to a match between a facial image in the stored gallery and the received facial image having a predetermined level of match certainty, identify the test subject; and
in response to a failure to obtain a match between a facial image in the stored gallery and the received facial image having a predetermined level of match certainty, flag the received facial image for subsequent manual analysis.

6. The system of claim 5, further comprising a vehicle interlock system including:
a camera for generating the facial image of the test subject; and
a wireless modem for transmitting the facial image to the facial recognition processing system.

7. The system of claim 6, wherein the vehicle interlock system comprises a test apparatus for testing the test subject for the selected substance and the facial recognition processing system is further operable to enable the test apparatus in advance of testing the test subject in response to an identification of the test subject.

8. A method of controlling a vehicle sobriety interlock system comprising:
taking a facial image of a vehicle operator with a camera forming a portion of a vehicle sobriety interlock system;
comparing the facial image of the vehicle operator with a set of facial images stored in a database with a processing system executing a facial recognition algorithm; and
in response to a match of a predetermined level of certainty between the facial image of the vehicle operator and a facial image of the set of images selectively activating a test device forming a portion of the vehicle sobriety interlock system for testing the vehicle operator for consumption of a selected substance.

9. The method of claim 8, wherein the processing system is remote from the vehicle sobriety interlock system and the method further comprises transmitting the image of the vehicle operator from a wireless modem forming a portion of the vehicle interlock system to the processing system.

10. The method of claim 8, wherein comparing the image of the vehicle operator with a set of images stored in a database comprises:
determining if the image of the vehicle operator sufficiently represents a human face; and
in response to a determination that the image of the vehicle operator sufficiently represents a human face, comparing the facial image of the vehicle operator with a set of facial images stored in a database with the processing system executing the facial recognition algorithm.

11. The method of claim 8, wherein comparing the image of the vehicle operator with a set of images stored in a database comprises:
determining if the image of the vehicle operator sufficiently represents a human face;
in response to a determination that the image of the vehicle operator does not sufficiently represent a human face, requiring a retake of a facial image of the vehicle operator; and
in response to the retake of the facial image of sufficiently representing a human face, comparing the facial image of the vehicle operator from the retake with a set of facial images stored in a database with the processing system executing the facial recognition algorithm.

12. The method of claim 8, further comprising:
in response to a failure to obtain match of a predetermined level of certainty between the facial image of the vehicle operator and a facial image of the set of images, flagging the image of the vehicle operator with the processing system for subsequent manual review.

13. The method of claim 8, further comprising:
in response to a failure to obtain match of a predetermined level of certainty between the facial image of the vehicle operator and a facial image of the set of images, requiring a retake of a facial image of the vehicle operator.

14. The method of claim 8, further comprising:
in response to a match of a predetermined level of certainty between the facial image of the vehicle operator and a facial image of the set of images, determining if the vehicle operator requires testing for consumption of the selected substance;
in response to a determination that the vehicle operator requires testing for consumption of the selected substance, activating the test device; and
in response to a determination that the vehicle operator does not require testing for consumption of the selected substance, bypassing the testing device to allow vehicle ignition.

15. A system for controlling operation of a vehicle having a vehicle sobriety interlock system comprising:
a camera forming a portion of a vehicle sobriety interlock system for taking a facial image of a vehicle operator;
a database storing a set of facial images; and
a processing system operable to:
execute a facial recognition algorithm to compare the facial image of the vehicle operator with the set of facial images stored in a database; and
in response to a match of a predetermined level of certainty between the facial image of the vehicle operator and a facial image of the set of images selectively activating a test device forming a portion of the vehicle sobriety interlock system for testing the vehicle operator for consumption of a selected substance.

16. The system of claim 15, wherein the processing system is remote from the vehicle sobriety interlock system and the vehicle sobriety interlock system further comprises a wireless modem for transmitting the facial image of the vehicle operator to the processing system.

17. The system of claim 15, wherein the testing device comprises a breath testing device for detecting the presence of alcohol in the breath of the vehicle operator.

18. The system of claim 15, wherein the processing system is operable to:
in response to a failure to obtain match of a predetermined level of certainty between the facial image of the vehicle operator and a facial image of the set of images, flagging the image of the vehicle operator for subsequent manual review.

19. The system of claim 15, wherein the predetermined level of certainty is programmable in the processing system by an administrator to set a level of subsequent manual analysis and a corresponding level of test circumvention protection.

20. The system of claim 15, wherein the processing system is further operable to:
in response to a failure to obtain match of a predetermined level of certainty between the facial image of the vehicle operator and a facial image of the set of images, requiring a retake of a facial image of the vehicle operator with the camera.

21. A method of controlling a vehicle sobriety interlock system comprising:
storing at least one first facial image in a database;
taking a second image with a camera forming a portion of a vehicle sobriety interlock system of an object;
executing a facial detection algorithm with a processor using the at least one facial image and the second image to determine if the object is a human face;
in response to a determination that the object is a human face, enabling the vehicle sobriety interlock system for testing the vehicle operator for consumption of a selected substance; and
in response to a determination that the object is not a human face, maintaining the vehicle sobriety interlock system in a disabled state.

22. The method of claim 21, further comprising, in response to a determination that the object is not a human face:
taking a third image with a camera forming a portion of a vehicle sobriety interlock system of the object;
executing a facial detection algorithm with a processor using the at least one facial image and the third image to determine if the object is a human face;
in response to a determination that the object is a human face, enabling the vehicle sobriety interlock system for testing the vehicle operator for consumption of a selected substance; and
in response to a determination that the object is not a human face, maintaining the vehicle sobriety interlock system in a disabled state.

23. The method of claim 21, wherein executing a facial detection algorithm with a processor comprises executing a facial detection algorithm with a processor forming a portion of the vehicle interlock system.

24. The method of claim 21, wherein executing a facial detection algorithm with a processor comprises executing a facial detection algorithm with a processor remote from the vehicle interlock system.

25. A method of testing for the use of a selected substance by an individual comprising:
during a predetermined test time window, collecting an air sample through an intake of a breath testing unit capable of measuring an amount of the selected substance in the air sample;
during collecting of the air sample, taking an image of an area proximate the intake of the breath testing unit with a camera; and
with a processing system:
processing measurement data received from the breath testing unit to determine whether the amount of the selected substance in the air sample exceeds a predetermined threshold;
processing image data received from the camera with an algorithm capable of detecting a human face from the image data received from the camera with a predetermined degree of confidence;
if the amount of the selected substance in the air sample exceeds the predetermined threshold, recording the substance test as failed;
if the amount of the selected substance in the air sample does not exceed the predetermined threshold and a human face is not detected in the image data received from the camera, recording the test as failed.

26. The method of claim 25, further comprising:
with the processing system:
if the amount of the selected substance in the air sample exceeds the predetermined threshold, determining if a maximum number of allowed breath retests have been taken; and
if the maximum number of allowed breath retests have not been taken, re-opening the test time window and allowing a retest for use of the selected substance.

27. The method of claim 25, further comprising:
with the processing system:
if a human face was not detected in the image data received from the camera, determining if a maximum number of allowed facial recognition attempts have been taken; and
if the maximum number of allowed facial recognition attempts have not been taken, re-opening the test time window and allowing a retest for use of the selected substance.

28. The method of claim 25, further comprising:
collecting air sample testing pass/fail results and facial detection pass/fail results with the processing system; and
transmitting the collected air sample testing and facial detection results to a server; and
generating a report including the air sample testing and facial detection results with the server and transmitting the report to a monitoring authority.

29. The method of claim 25, wherein the selected substance is alcohol.

30. The method of claim 25, further comprising:
transmitting the image to a remote server; and
performing facial recognition processing on the image data with the server to confirm an identity of a person providing the air sample.

31. The method of claim 25 further comprising:
performing an additional review of pictures received by a server from the breath testing unit when a facial detection certainty score falls between two predetermined thresholds.

32. A system for testing an individual for consumption of a selected substance comprising:
a breath testing unit for measuring an amount of the selected substance in an air sample received at an air intake;
a camera for taking an image of an area proximate the air intake during the collection of the air sample; and
a least one processor operable to:
process measurement data generated by the breath testing unit to determine whether the amount of the selected substance in the air sample exceeds a predetermined threshold; and
process image data received from the camera with a facial detection algorithm capable of detecting a human face in the received image data with a predetermined degree of confidence;
wherein the individual passes a test for consumption of the selected substance when the at least one processor determines that the amount of the selected substance in the air sample is below the preselected threshold and the at least one processor detects a human face in the image data received from the camera.

33. The system of claim 32, wherein the at least one processor comprises:
a first processor operable to process the measurement data generated by the breath testing unit and determining whether the amount of the selected substance in the air sample exceeds a predetermined threshold; and
a second processor operable to process the image data received from the camera with a facial detection algorithm capable of detecting a human face in the received image data with a predetermined degree of confidence;
wherein the individual passes a test for consumption of the selected substance when the first processor determines that the amount of the selected substance in the air sample is below the preselected threshold and the second processor detects a human face in the image data received from the camera.

34. The system of claim 32, wherein the at least one processor comprises a single processor operable to:
execute a first thread for processing the measurement data generated by the breath testing unit and determine whether the amount of the selected substance in the air sample exceeds a predetermined threshold; and
execute a second thread for processing the image data received from the camera with a facial detection algorithm capable of detecting a human face in the received image data with a predetermined degree of confidence;

35. The system of claim 32, further comprising a subsystem for transmitting air sample test results generated by the first processor and facial detection results generated by the second processor to a remove server.
